(19) United States
(12) Patent Application Publication
(10) Pub. No.: US 2018/0305453 A1
(43) Pub. Date: Oct. 25, 2018

(54) Monovalent Chimeras

(71) Applicant: CANADIAN BLOOD SERVICES, Ottawa (CA)

(72) Inventors: Alan H. Lazarus, Toronto (CA); Xiaojie Yu, Southampton (GB); William Peter Sheffield, Hamilton (CA)

(73) Assignee: Canadian Blood Services, Ottawa (CA)

(21) Appl. No.: 15/769,697

(22) PCT Filed: Oct. 20, 2016
(86) PCT No.: PCT/CA2016/051217

§ 371 Date: Apr. 19, 2018

Related U.S. Application Data
(60) Provisional application No. 62/244,769, filed on Oct. 22, 2015.

(51) Int. Cl. C07K 16/28; A61P 37/06

(52) U.S. Cl. CPC C07K 16/283; A61P 37/06; C07K 2317/35; C07K 2317/52; A61K 2039/505; C07K 2317/622; C07K 2317/55; C07K 2317/24; C07K 2319/31; C07K 2317/76

(57) ABSTRACT

The present disclosure relates to monovalent antibodies and chimeric proteins (comprising the monovalent antibodies) for the treatment of an auto-immune inflammatory disorder or condition. The monovalent antibody moiety lacks a Fc region, is specific for an activating Fc receptor and is for limiting or avoiding the activation of an immune cell induced in the presence and upon the binding of a ligand of the activating Fc receptor to the activating Fc receptor. The monovalent antibodies and chimeric proteins are especially useful for the prevention, treatment or alleviation of symptoms associated with an auto-immune inflammatory disorder caused or maintained by the engagement of an auto-antibody having a Fc region capable of engaging the activating Fc receptor to mediate the pathological destructions of cells or tissues.

### CROSS-REFERENCE TO RELATED APPLICATIONS AND DOCUMENTS

This application claims priority from U.S. provisional patent application 62/244,769 filed on Oct. 22, 2016 and herewith incorporated in its entirety. This application also includes a sequence listing in electronic format which is also incorporated in its entirety.

### TECHNOLOGICAL FIELD

This disclosure relates to monovalent antibodies specific for an activating Fc receptor as well as chimeric proteins comprising same for the use in the prevention, treatment and/or the alleviations of symptoms associated with an auto-immune inflammatory condition or disorder in a subject.

### BACKGROUND

Antibody-mediated pathological destruction of (self) cells or tissues is a major concern in the prevention and treatment of various auto-immune inflammatory conditions, such as, immune thrombocytopenia, rheumatoid arthritis, multiple sclerosis, type I diabetes, lupus erythematosus and hemolytic anemias. Antibodies which specifically recognize and bind to self-structures (such as cells and tissues) are recognized by the Fc receptor which is found on the surface of certain immune cells (among others, B lymphocytes, follicular dendritic cells, natural killer cells, macrophages, neutrophils, eosinophils, basophils and mast cells). The formation of a complex between auto-antibodies, the self structure and the Fc receptor contribute to the destruction of such self-structures by stimulating phagocytosis or antibody-dependent cell-mediated cytotoxicity against the “self” structures.

Immune thrombocytopenia (ITP) has been used as a model for studying antibody-mediated destruction of cells and tissues occurring in auto-immune conditions and disorders. In ITP, auto-immune anti-platelet antibodies cause the destruction of platelets. Antibody-mediated platelet destruction in the majority of ITP patients involves Fc-mediated phagocytosis by macrophages via the Fc gamma receptors (FcγRs). One of the major activating FcγRs implicated in platelet depletion is the FcγRIIIA, also a therapeutic target. The first FcγRIIIA-specific monoclonal antibody (mAb) 3G8 was described in 1982, and was later investigated clinically in ITP patients. Encouragingly, more than 50% of ITP patients refractory to other treatments responded with significantly improved platelet counts. However, its continued therapeutic application was stalled by adverse events, including vomiting, nausea and fever.

One potential means of reducing unwanted adverse events involves abolishing Fc-mediated effector function. A deglycosylated version of 3G8 (called GMA161), known to have abrogated Fc function, had thus been developed. In a humanized mouse model, GMA161 was able to ameliorate ITP, but unfortunately rapidly depleted granulocytes. Consistent with the humanized mouse model, GMA161 improved platelet counts in refractory patients but failed to reverse adverse events exhibited by its parent 3G8. Also, the Fab fragment of the anti-huFcγRIIIA 3G8 had been shown to be ineffective in ameliorating ITP in refractory patients.

It would be desirable to be provided with alternative therapeutics for the prevention, treatment or alleviation of symptoms of auto-immune inflammatory disorders or conditions caused or maintained by auto-antibodies which recognize and engage an activating Fc receptor. Preferably, the therapeutics would exhibit less unwanted side effects than existing therapeutics for example those observed with the 3G8 antibody or its de-glycosylated variant.

### SUMMARY

The present disclosure concerns chimeric proteins which includes a monovalent antibody moiety specifically recognizing and binding to an activating Fc receptor and is adaptable to be associated with a carrier. The monovalent antibody moiety does not have (e.g., it lacks) a Fc region. The monovalent antibody moiety is especially useful for limiting or avoiding the activation of an immune cell caused or induced by the presence and the binding of a ligand of the activating Fc receptor to the activating Fc receptor. When the monovalent antibody moiety is presented as a chimeric protein comprising a carrier, the latter is at least 40 kDa, is physiologically acceptable and does not induce or trigger a pro-inflammatory response. The monovalent antibodies and chimeric proteins can be used in the prevention, treatment or alleviation of symptoms of an auto-immune diseases or disorders.

In a first aspect, the present disclosure provides a monovalent antibody moiety optionally associated with a carrier. The monovalent antibody moiety lacks a Fc region. The monovalent antibody moiety is also capable of specifically binding to a component of an activating Fc receptor. In an embodiment, the monovalent antibody is a competitive inhibitor of the activating Fc receptor. In another embodiment, the monovalent antibody is an allosteric inhibitor of the activating Fc receptor. In still a further embodiment, the monovalent antibody moiety is a single chain variable fragment (scFv). In still another embodiment, the monovalent antibody moiety is a fragment antigen-binding (Fab). In an embodiment, the monovalent antibody moiety can be derived from a 3G8 antibody or a 2.4G2 antibody. In a further embodiment, the component of the activating Fc receptor is a FcγR receptor and, in yet a further embodiment, the component of the activating Fcγ receptor is a FcγRIII polypeptide.

In a second aspect, the present disclosure provides a chimeric protein comprising the monovalent antibody described herein and a carrier. The carrier is physiologically acceptable.

The carrier also lacks the ability to induce a pro-inflammatory immune response. The carrier has a molecular weight equal to or greater than 40 kDa. In another embodiment, the monovalent antibody moiety is covalently associated to the carrier, either directly or indirectly (via a linker). In another embodiment, the monovalent antibody moiety is non-covalently associated to the carrier, either directly or indirectly (via a linker). In a further embodiment, the chimeric protein further comprises a linker (such as, for example an amino acid linker or an antibody-derived linker) between the monovalent antibody moiety and the carrier. In another embodiment, the carrier is a polypeptide, such as, for example, a blood protein such as, for example albumin. In an embodiment, the carboxyl terminus of the monovalent antibody moiety is associated to the carrier. In yet another embodiment, the carrier is a polypeptide and the amino terminus of the carrier is associated to the carboxyl terminus of the monovalent antibody moiety.

In a third aspect, the present disclosure provides a monovalent antibody moiety or a chimeric protein as defined herein for use as a medicament or in therapy.

In a fourth aspect, the present disclosure provides a monovalent antibody moiety or a chimeric protein as defined herein for the prevention, treatment or alleviation of symptoms of an auto-immune inflammatory condition or disorder caused or maintained by the engagement of an auto-antibody having a Fc region capable of engaging to an activating Fc receptor of an immune cell of the subject. The present disclosure also provides the use of a chimeric protein as defined herein for the prevention, treatment or alleviation of symptoms of an auto-immune inflammatory condition or disorder caused or maintained by the engagement of an auto-antibody having a Fc region capable of engaging to an activating Fc receptor of an immune cell of the subject. The present disclosure further provides the use of a monovalent antibody moiety or a chimeric protein as defined herein for the manufacture of a medicament for the prevention, treatment or alleviation of symptoms of an auto-immune inflammatory condition or disorder caused or maintained by the engagement of an auto-antibody having a Fc region capable of engaging to an activating Fc receptor of an immune cell of the subject. In an embodiment, the auto-immune inflammatory condition or disorder is immune cytopenia such as, for example, idiopathic immune thrombocytopenia or autoimmune hemolytic anemia (AHA).

In a fifth aspect, the present disclosure provides a method for preventing, treating or alleviating the symptoms of an auto-immune inflammatory condition or disorder caused or maintained by engagement of an auto-antibody having a Fc region capable of engaging to an activating Fc receptor of an immune cell of a subject. The method comprises administering a therapeutically effective amount of a monovalent antibody moiety or a chimeric protein as defined herein so as to prevent, treat or alleviate the symptoms of the auto-immune inflammatory condition or disorder in the subject. In an embodiment, the auto-immune inflammatory condition or disorder is immune cytopenia such as, for example, idiopathic immune thrombocytopenia or autoimmune hemolytic anemia (AHA).

### BRIEF DESCRIPTION OF THE DRAWINGS

FIGS. 1A and B. In vitro binding activity of 3G8 scFv-HSA for huFcγRIIIA. Binding of 3G8 scFv-HSA fusion protein to the soluble domain of huFcγRIIIA was assessed by enzyme-linked immunosorbent assay. High binding plate was coated with recombinant huFcγRIIIA overnight. (A) To detect direct binding of 3G8 scFv-HSA to huFcγRIIIA (◯), or HSA (□) (highest concentration: 870 nM) was added, and bound 3G8 scFv-HSA was detected by anti-HSA-HRP. n=6, data representative of 3 independent experiments. (B) To assess the ability of 3G8 scFv-HSA to competitively inhibit huIgG binding to huFcγRIIIA, various concentrations of 3G8 scFv-HSA (◯, highest concentration: 650 nM), HSA (Δ, highest concentration: 650 nM), 3G8 (□, highest concentration: 67 nM) or vehicle (∇) to wells containing 0.8 μg/mL huIgG. n=4, data representative of 5 independent experiments. All data points represented as mean±SEM.

FIGS. 2A to C. In vitro binding activity of 2.4G2 scFv-MSA for murine FcγRIII/IIB and in vivo pharmacokinetics. (A) RAW264.7 macrophage-like cell line, known to express FcγRIII and FcγRIIB, were stained with 0.11 μM 2.4G2 scFv-MSA (10 μg/mL) in the presence of vehicle control (PBS) or equimolar amount of 2.4G2 or HSA (as competitive inhibitors). Residual bound 2.4G2 scFv-MSA was detected by anti-His-PE. Data representative of 4 independent experiments. (B) To analyze the ability of 2.4G2 scFv-MSA to inhibit PE-labeled 2.4G2 binding, RAW264.7 cells were stained with 0.013 μM (2 μg/mL) PE-labeled 2.4G2 in the presence of 0.11 μM (10 μg/mL) 2.4G2 scFv-MSA, HSA, 2.4G2 or PBS. Data representative of 5 independent experiments. (C) For in vivo pharmacokinetics analysis, mice were injected with 80 μg 2.4G2 scFv-MSA or approximately 200 μg 2.4G2 Fab, and then bled after 0.5, 2, 4, 8, 24 and 48 hours. Serum samples were prepared and used to stain RAW264.7 cells; bound 2.4G2 scFv-MSA was detected by anti-His-PE, and bound 2.4G2 Fab in serum were detected by anti-rat IgG-κ chain-PE. Level of remaining serum protein was expressed as a percentage of MFI at 0.5 hr. n=6-8, from 3 independent experiments. Data are presented as mean±SEM.

FIGS. 3A and B. In vivo efficacy of 2.4G2 scFv-MSA in ITP amelioration. (A) Mice were pretreated intravenously with 10 (⋄), 20 (◯), 40 (Δ) or 80 (∇) μg of 2.4G2 scFv-MSA or 56 μg HSA (□, equimolar amount as 80 μg 2.4G2 scFv-MSA) for 2 hours before ITP induction by administration of 2 μg anti-platelet antibody MWReg30. Mice were then bled after 2, 24 and 48 hours and platelets were enumerated using a Z2 particle counter. ***p<0.01 compared with HSA at each time point, n=6-8, from 4 independent experiments. (B) Mice were pretreated with 25 mg IVIg (◯, intraperitoneally), 80 μg 2.4G2 scFv-MSA (∇) or 56 μg HSA (□) for 2 hours before ITP induction by administration of 3 μg anti-platelet antibody 6A6. Mice were then bled after 2, 24 and 48 hours and platelets were enumerated using a Z2 particle counter. n=6-7, from 4 independent experiments.

FIG. 4. 2.4G2 antibody and 2.4G2 scFv-MSA induced changes in body temperature. Mice were treated with 0.43 nmol (65 μg) 2.4G2 (◯) or equimolar amount of 2.4G2 scFv-MSA (∇) or HSA. Cross-linked 2.4G2 scFv-MSA (⋄) was prepared by mixing 0.43 nmol 2.4G2 scFv-MSA and half-molar anti-His monoclonal antibody (Δ) for 30 minutes at room temperature. Body (rectal) temperature was measured 0.5, 1, 1.5 and 2 hours after treatment by a thermometer. n=6-9, from 3 independent experiments.

FIGS. 5A to C. 2.4G2 antibody and 2.4G2 scFv-MSA induced basophil activation. (A) To analyze CD200R3 levels on basophils, RBCs in peripheral blood was lysed by ammonium chloride buffer before staining with anti-CD49b-Pacific Blue™, anti-FcεRIα-PerCP/Cy5.5 and anti-CD200R3-FITC. The population within gate P1 represents PBMCs (left panel), and was further gated based on CD49b and FcεRIα expression levels. The population within P2 (middle panel) represents basophils (P2 shown in FCS and SSC plot, right panel), evidenced by (B) expression of CD200R3. (C) Mice were bled before treatment, 4, and 24 hours after administration of 0.43 nmol (65 μg) 2.4G2 or equimolar amount of 2.4G2 scFv-MSA or HSA. Samples were stained with anti-CD49b-Pacific Blue™, anti-FcεRIα-PerCP/Cy5.5 and anti-CD200R3-FITC. All samples were analyzed by MACS Quant. Data were analyzed by Flowjo V10 Software. Dot plots and histograms representative of 6-7 mice per group from 4 independent experiments.

FIG. 6. 2.4G2 antibody and 2.4G2 scFv-MSA induced transient basophil depletion. Mice were bled before treatment, 4, and 24 hours after treatment with 0.43 nmol (65 μg) 2.4G2 or equimolar amount of 2.4G2 scFv-MSA or HSA. Ammonium chloride buffer was used to lyse RBCs before PBMCs were stained with anti-CD49b-Pacific Blue™, anti-FcεRIα-PerCP/Cy5.5 and anti-CD200R3-FITC. Stained samples were analyzed by MACS Quant and data were analyzed by Flowjo V10 Software. Basophils were identified as CD49 dim, FcεRIα positive and CD200R3 positive. An oval gate is used to mark basophil population. The frequency represents the percentage of basophils within the whole PBMC population shown as P1 in FIG. 5A. Basophil concentrations (per microliter blood) represent in vivo concentrations. Histograms representative of 6-7 mice per group from 4 independent experiments.

FIG. 7. 2.4G2 scFv-MSA improves cbc512-mediated autoimmune hemolytic anemia (AHA). The anti-RBC mAb cbc512 (9 μg) was injected on day 0. Twenty-four (24) hours later, mice were treated with 150 μg 2.4G2 scFv-murine serum albumin (MSA) (▴) or equimolar amount of HSA (▪) as control. Control animals having received PBS are shown as •. Red blood cell count was enumerated on 48 and 72 hours. n=6-8; from 3 independent experiments.

### DETAILED DESCRIPTION

The present disclosure provides a monovalent antibody moiety, optionally in combination with a carrier to form a chimeric protein. The terms “chimeric protein” or “chimera” refer to a first proteinaceous entity (e.g., a monovalent antibody moiety) which is associated with another (second) entity, which may be proteinaceous as well. The first proteinaceous entity does not naturally occur in association with the second entity. The first proteinaceous entity is modified (via genetic or chemical means) to be capable of associating or be associated with the second entity. The first and second entity may be derived from the same species or the same genera or can be derived from different species or different genera. The first and second entity can be derived from the genera or the species intended to receive the monovalent antibody or the chimeric protein. For example, the first and/or the second entity can be derived from humans if the monovalent antibody or the chimeric protein are intended to be administered to humans.

The chimeric protein comprises at least two components or entities: a monovalent antibody moiety and a carrier. The two entities can be associated together prior to the administration to a recipient. The two entities can also be associated only after the monovalent antibody moiety is administered to the recipient. The association between the two moieties can be covalent or non-covalent and can occur prior to, during or after administration.

In the chimeric proteins of the present disclosure, the monovalent antibody moiety is associated to a carrier. The term “carrier”, as used herein, refers to a molecule that is capable of being associated (covalently or non-covalently, directly or indirectly) with the monovalent antibody. The carrier is physiologically acceptable. The carrier also lacks the ability of eliciting a pro-inflammatory response, e.g., the carrier, much like the linker, does not participate to the inflammatory process nor does it elicit the production of antibodies recognizing the chimeric protein. In an embodiment, the carrier is immunologically inert, e.g., it lacks the ability to elicit an immune response. In another embodiment, the carrier has the ability to elicit an anti-immunogenic response or a pro-tolerogenic immune response. The carrier does not bind directly to the activating Fc receptor nor does not cause the chimeric protein to bind to more than one site on the activating Fc receptor. The carrier does not cause the association of two or more chimeric proteins to simultaneously bind more than one site on the activating Fc receptor. The carrier does not substantially interfere with the binding specificity and/or affinity of the monovalent antibody moiety of the chimeric protein. In certain conditions, the carrier can modestly lower the binding affinity of the monovalent antibody moiety present in the chimeric protein when compared to the free from monovalent antibody moiety (not included in a chimeric protein). Still preferably, the carrier has a longer clearance time in the blood stream than the monovalent antibody moiety alone. It is known in the art that carriers having a molecular weight equal to or higher than 40 kDa (or even higher than 60 kDa) are less rapidly expelled by the kidney and, consequently, have a longer half-life in blood than molecules or smaller size (such as the monovalent antibody moiety described herein). In an embodiment, the carrier has the ability to bind to the neonatal Fc receptor (also referred to as FcRn) to increase the presence of the chimeric protein in plasma. For example, the carrier can be albumin or an antibody fragment (lacking its Fc moiety) specifically recognizing the FcRn.

In an embodiment, the carrier is a protein or polypeptide, such as, for example, a plasma protein. Plasma proteins include, but are not limited to serum albumin, immunoglobulins fragments (provided that these fragments do not directly bind the activating Fc receptor or cause the chimeric protein to simultaneously bind to more than one site on the activating Fc receptor), alpha-1-acid glycoprotein, transferrin, or lipoproteins. In some instances, it is contemplated that a human protein, such as a human plasma protein be used as the carrier. This embodiment is particularly useful when designing therapeutics for the treatment of humans or for making a chimeric protein in which the monovalent antibody moiety is derived (directly or indirectly) from a human antibody or a humanized antibody. In an embodiment, the carrier is immunoglobulin fragment, such as a monovalent antibody moiety of an antibody, for example the anti-neonatal FcR (FcRn) antibody. In such embodiment, the antibody-binding region of the anti-FcRn antibody is associated with the monovalent antibody in order to allow the recognition and binding of the carrier to the FcRn. In another embodiment, the carrier is not proteinaceous in nature, but is rather a chemical polymer. Such polymers include, but are not limited to, PEG.

In some instances, the chimeric protein is exclusively made of amino acids and is produced by a living organism using a genetic recombination technique. The chimeric protein can consist of a monovalent antibody moiety (preferably specific for the Fcγ receptor), albumin as a carrier and an amino acid linker (such as, for example, a multi-glycine linker (G6 linker)).

In the chimeric protein, the monovalent antibody moiety can be associated directly to the carrier. Alternatively, the monovalent antibody moiety can be associated indirectly to the carrier by using one of more linkers between the monovalent antibody moiety and the carrier. Preferably a single linker is used to indirectly associate the monovalent antibody moiety and the carrier. In the context of the present disclosure, the linker must be selected so as not to cause the production of specific antibodies or be recognized by existing antibodies upon the administration to the subject. In an embodiment, the linker is composed of one or more amino acid residues located between the monovalent antibody moiety and the carrier. This embodiment is especially useful when the chimeric protein is intended to be produced in a living organism using a genetic recombinant technique. The amino acid linker can comprise one or more amino acid residues. For example, the amino acid linker can comprises one or more glycine residues such as an hexa-glycine linker. The present chimeric protein also includes those using a non-amino acid linker, such as a chemical linker.

The monovalent antibody moiety can be associated with the linker or the carrier at any amino acid residue(s), provided that the association does not impede the monovalent antibody moiety from binding to the activating Fc receptor. In some instances, the linker or the carrier is associated to one or more amino acid residue(s) of the monovalent antibody moiety that is (are) not involved in specifically binding the activating Fc receptor. In some instances, the linker or the carrier is associated to a single amino acid residue of the monovalent antibody moiety. The linker or the carrier can be associated with any amino acid residue of the monovalent antibody moiety, including the amino acid residue located at the amino-terminus of the monovalent antibody moiety or at the carboxyl-terminus of the monovalent antibody moiety. In instances in which the linker and the carrier are also of proteinaceous nature, the monovalent antibody moiety can be associated to any amino acid residue of the linker or the carrier, including the amino acid residue located at the amino-terminus of the linker or the carrier or the amino acid residue located at the carboxyl-terminus of the linker or the carrier. In an embodiment, the amino acid residue located at the amino-terminus of the linker or the carrier is associated to the amino acid residue located at the carboxyl-terminus of the monovalent antibody moiety. In still another embodiment, when the linker is present and is of proteinaceous nature, its amino terminus is associated to the carboxyl terminus of monovalent antibody and its carboxyl terminus is associated with the amino terminus of the carrier.

In instances where a covalent association is sought between the monovalent antibody moiety and the carrier, the association between the two entities can be a peptidic bond. Such embodiment is especially useful for chimeric proteins wherein the at least two entities are both proteinaceous and are intended to be produced as a fusion protein in an organism (prokaryotic or eukaryotic) using a genetic recombinant technique. Alternatively, the covalent association between the two moieties can be mediated by any other type of chemical covalent bounding. In some instances, the chimeric proteins are designed so as not to be susceptible of being cleaved into the two moieties in the general circulation (for example in plasma).

As indicated above, the association between the two entities can be non-covalent. Exemplary non-covalent associations include, but are not limited to the biotin-streptavidin/avidin system. In such system, a label (biotin) is covalently associated to one entity/moiety while a protein (streptavidin or biotin) is covalently associated with the other entity/moiety. In such embodiment, the biotin can be associated to the monovalent antibody moiety or to the carrier, providing that the other entity in the system is associated with streptavidin or avidin.

In a further system of non-covalent association, the first entity is designed to be non-covalently associated to the second entity only upon its administration into the intended recipient. This embodiment is especially useful when the carrier is a protein present in the blood of the recipient. For example, the monovalent antibody moiety may be associated (in a covalent or a non-covalent fashion) with a second antibody, a lectin or a fragment thereof (referred to herein as an antibody-derived linker) which is capable of non-covalently binding the carrier once administrated to the intended recipient. For example, the second antibody, lectin or fragment thereof can be specific for any blood/plasma protein present in the intended recipient (such as, for example, serum albumin, immunoglobulins fragments (provided that these fragments do not directly bind the activating Fc receptor or cause the chimeric protein to simultaneously bind to more than one site on the activating Fc receptor), alpha-1-acid glycoprotein, transferrin, or lipoproteins). The second antibody, lectin or fragment thereof can be associated, preferably in a covalent manner, with the monovalent antibody moiety at any amino acid residue of the monovalent antibody moiety, but preferably at the amino- or carboxyl-end of the monovalent antibody moiety. In such embodiment, the second antibody, lectin or fragment thereof is akin to a linker between the monovalent antibody moiety and the carrier. Upon the administration of this embodiment of the monovalent antibody moiety in the recipient, the carrier (a blood or plasma protein for example) associates with the second antibody, lectin or fragment thereof to form, in vivo, the chimeric protein. In a specific embodiment, the second antibody is an antibody specifically recognizing albumin (such as, for example, an antibody specifically recognizing human albumin).

In the present disclosure, the monovalent antibody moiety can be considered to be a competitive inhibitor of the activating Fc receptor. More specifically, the monovalent antibody moiety can compete with a binding site used by the activating Fc receptor ligand. The Fc receptor ligands are the Fc region of antibodies. Upon the binding of the Fc receptor ligands to the activating Fc receptor, the activating. Fc receptor cross-links and mediates an internal signaling leading to a pro-inflammatory immune response in an immune cell. As such, when the monovalent antibody moiety or the chimeric protein is a competitive inhibitor of the activating Fc receptor, it competes for the activating Fc receptor ligand's binding site(s) and either prevents the activating Fc receptor ligand from binding to the activating Fc receptor or limits the amount of the Fc receptor ligand that can bind to the activating Fc receptor.

Alternatively, the monovalent antibody moiety or the chimeric protein comprising same can be considered to be an allosteric inhibitor of the activating Fc receptor. In such embodiment, the monovalent antibody moiety does not bind to a binding site used by the Fc receptor ligand. Instead, the monovalent antibody moiety binds to another binding site on the activating Fc receptor which alters the conformation of the activating Fc receptor and limits or prevent the binding of the Fc receptor ligand to the activating Fc receptor. As such, when the monovalent antibody moiety or the chimeric protein comprising same is an allosteric inhibitor of the activating Fc receptor, it binds to the activating Fc receptor on a site which is not involved with binding to the Fc receptor ligand and either prevents the ligand from binding to the activating Fc receptor or limits the amount of ligand that can bind to the activating Fc receptor (through presumably a conformational change in the receptor).

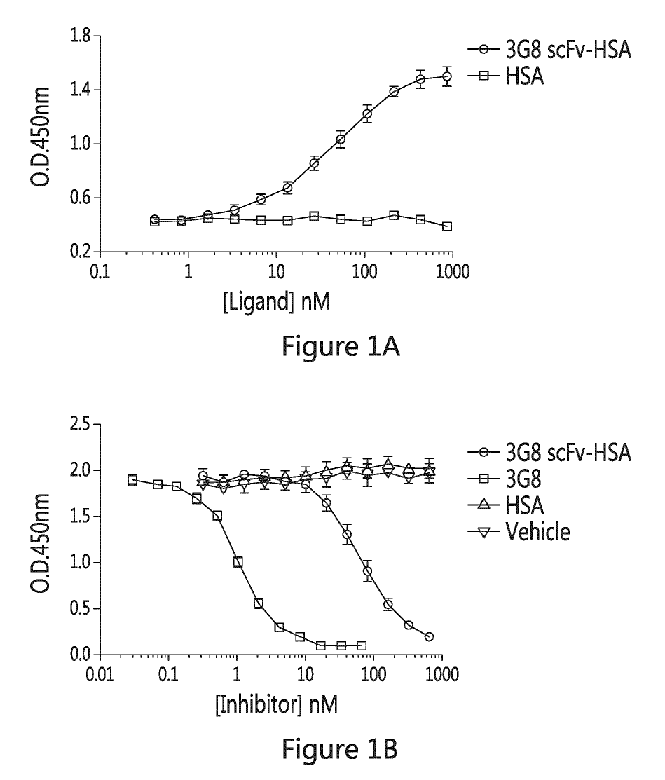

The monovalent antibody moiety can be derived (directly or indirectly) from a multivalent antibody. The monovalent antibody moiety is capable of competing for the binding site that is recognized by the corresponding multivalent antibody (see FIG. 1). The monovalent antibody moiety does not include the crystallizable fragment (Fc fragment) of the multivalent antibody it is derived from. The monovalent antibody moiety can be derived (directly or indirectly) from antibodies of any isotypes including IgA, IgD, IgE, IgG, IgM, IgW or IgY. The monovalent antibody can be derived from more than one antibody or from more than one genera or species and, in such instances, is characterized as being a chimeric monovalent antibody moiety. In some instances, the monovalent antibody moiety is derived (directly or indirectly) from the IgG antibody and preferably from a human IgG antibody. The antibody moiety is considered to be “monovalent” because it contains a single antigen binding site. The monovalent antibody moiety has no more than three variable light domains (V_{L}) associated (covalently or not) and no more than three corresponding variable heavy domains (V_{H}). This contrasts with multivalent full-length antibodies which comprises at least two antigen binding sites and more than three V_{H} and more than three V_{L} domains. The monovalent antibody moiety can be fully or partially glycosylated, when compared to the parent multivalent antibody it can be derived from. In some instances, the monovalent antibody moiety is not glycosylated. The monovalent antibody moiety can be a humanized or a chimeric monovalent antibody moiety.

In some instances, the monovalent antibody is a single-chain variable fragment (scFv) derived from one or more multivalent antibody. The scFv is single molecular entity (a fusion protein) consisting of a single antigen-binding region and having no more than three V_{H} and no more than three V_{L} domains from a multivalent antibody which are connected with a linker (e.g., usually a short peptide linker). As such, the scFv consists of a single antigen-binding region and comprises three V_{L} and three V_{H} domains. The scFv can be obtained from screening a synthetic library of scFvs, such as, for example, a phage display library of scFvs.

In other instances, the monovalent antibody moiety is the fragment antigen-binding region (Fab) of a multivalent antibody. The Fab fragment comprises two molecular entities (a light chain fragment and a heavy chain fragment), consists of a single antigen-binding site and comprises one constant and one variable domain from each heavy and light chain of the antibody which are associated to one another by disulfide bonds. The Fab includes three V_{L} and three V_{H} domains.

The monovalent antibody moieties are capable of specifically binding to a component of the activating Fc receptor. The Fc receptor is a receptor present on the surface of various immune cells such as, for example, B lymphocytes, follicular dendritic cells, natural killer cells, macrophages, neutrophils, eosinophils, basophils and mast cells. The monovalent antibody moiety binds to and recognizes a single antigen or epitope on the activating Fc receptor, which can be located on the Fcα receptor, the Fcγ receptor or the Fcε receptor. When the monovalent antibody moiety specifically recognizes and binds to the activating Fcγ receptor, it can be specific for the FcγRI, the FcγRII (including the FcγRIIA, FcγRIIB1 and FcγRIIB2) or the FcγRIII (FcγRIIIA, FcγRIIIB) polypeptide. In an embodiment, the monovalent antibody specifically recognizes and binds to the FcγRIIIA polypeptide The epitope recognized by the monovalent antibody moiety can be located anywhere on the activating Fc receptor and is preferably a epitope located on the extracellular portion of the activating Fc receptor. In some embodiments, even though the monovalent antibody moiety lacks a Fc region, the monovalent antibody moiety can bind to the activating Fc receptor portion which does recognize the Fc portion of the Fc receptor ligands (antibodies). In alternative embodiments, the monovalent antibody moiety recognizes and binds to a single epitope of the activating Fc receptor which is not involved in binding the Fc receptor ligands. In an embodiment, the monovalent antibody moiety specifically recognizes and binds to a component of the Fcγ receptor. Components of the Fcγ receptor include, but are not limited to, FcγRI (CD64), FcγRIIA (CD32), FcγRIIB (CD32), FcγRIIIA (CD16a), FcγRIIIB (CD16b) or FcγRIV. In some specific embodiments, the monovalent antibody moiety recognizes and binds to the FcγRIIIA component of the Fcγ receptor. In another embodiment, the monovalent antibody moiety specifically recognizes and binds to a component of the Fcα receptor, such as, for example, FcαRI (CD89). In yet a further embodiment, the monovalent antibody moiety specifically recognizes and binds to a component of the Fcα/μ receptor. In still a further embodiment, the monovalent antibody moiety specifically recognizes and binds to a component of the Fcε receptor. Components of the Fcε receptor include, but are not limited to, FcεRI and FcεRII (CD23).

The monovalent antibody moiety is capable of limiting or avoiding the activation of an immune cell induced by the presence and binding of a ligand of the activating Fc receptor to the activating Fc receptor. In some embodiment, the monovalent antibody is capable of preventing signaling from the component of the activating Fc receptor. This can be achieved by the ability of the monovalent antibody moiety to prevent or limit the binding of the activating Fc receptor ligand to the activating Fc receptor, to prevent or limit the cross-linking the activating Fc receptor upon binding to the activating Fc receptor ligand and/or to prevent or limit signaling from the activating Fc receptor (for example signaling associated with a trigger of phagocytosis by the cell comprising the activating Fc receptor). For example, the monovalent antibody is capable of binding to the activating Fc receptor and either limit or prevent the binding of the Fc region of an antibody to bind to the activating Fc receptor and/or limit or prevent signaling from the activating Fc receptor upon the binding of the Fc region of antibody to the activating Fc receptor. Methods for determining the binding of the Fc receptor ligand to the activating Fc receptor or ability to block signaling from an activating Fc receptor are known to those skilled in the art, and include, for example, ELISA and FACS.

The chimeric protein can be used to prevent, treat or alleviate the symptoms associated with an auto-immune inflammatory condition or disorder. In the context of the present disclosure, the expression “inflammatory condition or disorder” refers to diseases in which inflammation is involved (either it creates the disease or maintains it). A cascade of biochemical events propagates and matures the inflammatory response, involving the local vascular system, the immune system, and various cells within the injured cells or tissues. Inflammatory conditions and disorders collectively refer to a dysregulated inflammatory response which causes a pathological cellular destruction of cells or tissues in an afflicted subject. The inflammation can either be acute or chronic. Acute inflammatory conditions include, but are not limited to sepsis and encephalitis. Chronic inflammatory conditions share several clinical features, including persistent activation of the innate and acquired immune systems. The chronic inflammatory conditions can include the production of pro-inflammatory cytokines (IL-1, IL-18, IL-12, IL-23) and mediators (leukotrienes), the release of toxic species (reactive oxygen radicals) and proteases (lysosomal enzymes). In some embodiments, the chronic inflammatory condition also includes recruiting and activating other myeloid and lymphoid cells from systemic sites, such as, for example, CD8+ and CD4+ T lymphocytes (Th1, Th2 and Th17 cells). Persistence of pro-inflammatory T helper programs in these cells (Th1, Th2, Th17) and/or defects in suppressive T regulatory (Treg) responses can lead to unrelenting tissue damage. The auto-immune inflammatory disorders or conditions of the present disclosure are caused or maintained by the engagement of the Fc region of auto-antibodies with an activating Fc receptor on the surface of immune cells. As such, the immune system of the subject intended to receive the chimeric protein described herein, makes antibodies which recognize self structures (such as proteins, cells or tissues) and target such self structure for immune-mediated destruction. Chronic auto-immune inflammatory conditions includes, but are not limited to, asthma, idiopathic immune thrombocytopenia (ITP), auto-immune hemolytic anemia (AHA), autoimmune neutropenia, rheumatoid arthritis (RA), inflammatory bowel disease (IBD), Crohn's disease (CD), systemic lupus erythematosus (SLE), psoriasis (PA), multiple sclerosis (MS), type 1 diabetes (T1D), and celiac disease (CeD). Other conditions associated with chronic inflammation include, but are not limited to chronic obstructive pulmonary disease, coronary atherosclerosis, diabetes, metabolic syndrome X, cancer and neurodegenerative disorders. Acute auto-immune inflammatory disorders or conditions also include allergic reactions such as anaphylaxis.

In some embodiments, it is possible to customize the chimeric protein to specifically target one kind of activating Fc receptor involved in a specific disease or condition. For example, it is known that idiopathic immune thrombocytopenia is caused, in some instances, by the presence of IgG antibodies specific for platelets which ultimately cause the phagocytosis of the opsonized platelets. As such, it is possible to design a chimeric protein comprising a monovalent antibody moiety specific for a Fcγ receptor (for example a monovalent antibody specific for a FcγRIIIA polypeptide) for the prevention, treatment or the alleviation of symptoms associated with idiopathic immune thrombocytopenia. As another example, it is known that asthma and allergic reactions are in part mediated by the presence of IgE antibodies opsonizing non-self antigens and triggering inflammation as well as the release of histamine. As such, it is possible to design a chimeric protein comprising a monovalent antibody moiety specific for a Fcε receptor (for example a monovalent antibody specific for a FcεRI polypeptide) for the prevention, treatment or alleviation of symptoms associated with asthma and allergic reactions.

In the example provided herein, in a mouse model of ITP (an exemplary auto-immune mediated by auto-antibody engaging the activating Fc receptor), it was shown that the administration of an embodiment of the chimeric protein described herein prevented the onset of the disease and failed to exhibit negative side effects usually encountered with a multivalent antibody (such as fever). In another example provided herein, in a mouse model of AHA (an exemplary cytopenia mediated by auto-antibody engaging the activating Fc receptor), it was shown that the administration of an embodiment of the chimeric protein described herein treated the disease and ameliorated the low erythrocyte counts observed in untreated mice. These results show that the monovalent chimeras can both prevent and treat these cytopenias. As such, the present disclosure concerns the use of the monovalent antibody or the chimeric protein comprising same for the prevention, treatment or alleviation of symptoms associated with an auto-immune disease which is caused, induced or maintained by the presence of antibodies. Auto-immune diseases which are maintained, mediated or induced by the antibodies are also considered inflammatory disorders. Such auto-immune disorders include, but are not limited to immune thrombocytopenia, rheumatoid arthritis, type 1 diabetes, multiple sclerosis, systematic lupus erythematosus, psoriasis, etc. Preferably, the immune thrombocytopenia is idiopathic and involves the destruction of platelets. In the context of the present disclosure, immune thrombocytopenia is not caused by a viral infection (an HIV infection for example).

The monovalent antibody or the chimeric protein comprising same can successfully be used as an anti-inflammatory agent to prevent, treat or ameliorate the symptoms associated with an auto-immune inflammatory condition or disorder. The monovalent antibody or the chimeric protein can be used alone or in combination with other known anti-inflammatory agents.

The monovalent antibody or the chimeric protein comprising same can be formulated for administration with an excipient. An excipient or “pharmaceutical excipient” is a pharmaceutically acceptable solvent, suspending agent or any other pharmacologically inert vehicle for delivering one or more chimeric protein to a subject, and is typically liquid. A pharmaceutical excipient is generally selected to provide for the desired bulk, consistency, etc., when combined with components of a given pharmaceutical composition, in view of the intended administration mode. Typical pharmaceutical excipients include, but are not limited to binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

The monovalent antibody or the chimeric protein comprising same may be formulated for administration with a pharmaceutically-acceptable excipient, in unit dosage form or as a pharmaceutical composition. Conventional pharmaceutical practice may be employed to provide suitable formulations or compositions to administer such compositions to subjects. Although intravenous administration is preferred, any appropriate route of administration may be employed, for example, oral, parenteral, subcutaneous, intramuscular, intracranial, intraorbital, ophthalmic, intraventricular, intracapsular, intraspinal, intrathecal, epidural, intracisternal, intraperitoneal, intranasal, or aerosol administration. Therapeutic formulations may be in the form of liquid solutions or suspension. Methods well known in the art for making formulations are found in, for example, Remington: The Science and Practice of Pharmacy, (19th ed.) ed. A. R. Gennaro A R., 1995, Mack Publishing Company, Easton, Pa.

In addition, the term “pharmaceutically effective amount” or “therapeutically effective amount” refers to an amount (dose) effective in treating a subject afflicted by or suspected to be afflicted by an auto-immune inflammatory condition or disorder. It is also to be understood herein that a “pharmaceutically effective amount” may be interpreted as an amount giving a desired therapeutic effect, either taken in one dose or in any dosage or route, taken alone or in combination with other therapeutic agents.

A therapeutically effective amount or dosage of the monovalent antibody or the chimeric protein comprising same disclosed herein or a pharmaceutical composition comprising the chimeras, may range from about 0.001 to 30 mg/kg body weight, with other ranges of the invention including about 0.01 to 25 mg/kg body weight, about 0.025 to 10 mg/kg body weight, about 0.3 to 20 mg/kg body weight, about 0.1 to 20 mg/kg body weight, about 1 to 10 mg/kg body weight, 2 to 9 mg/kg body weight, 3 to 8 mg/kg body weight, 4 to 7 mg/kg body weight, 5 to 6 mg/kg body weight, and 20 to 50 mg/kg body weight. In other embodiments, a therapeutically effective amount or dosage may range from about 0.001 to 50 mg total, with other ranges of the invention including about 0.01 to 10 mg, about 0.3 to 3 mg, about 3 to 10 mg, about 6 mg, about 9 mg, about 10 to 20 mg, about 20-30 mg, about 30 to 40 mg, and about 40 to 50 mg. In an embodiment, the chimera is administered to a dosage between about 40-80 mg/kg (e.g. 60 mg/kg).

The present invention will be more readily understood by referring to the following examples which are given to illustrate the invention rather than to limit its scope.

### EXAMPLE

Mice. CD-1 female mice (Charles River Laboratories, Kingston, N.Y., USA) and HOD (hen egg lysozyme, ovalbumin, and human Duffy^{b}) transgenic mice were used for the in vivo experiments. All mice were housed with water and food ad libitum. All animal experiments were approved by the St Michael's Hospital Animal Care and Use Committee.

Antibodies and reagents. Rat IgG2b-FITC isotype control was purchased from Miltenyi Biotech, Canada. Unconjugated monoclonal anti-His antibody (HIS.H8) and AmpliTaq Gold™ 360 Master Mix were from Life Technologies, Canada. The unconjugated murine FcγRIII/IIB-specific 2.4G2 was from BioX Cell, USA, and the Fab fragment of 2.4G2 was generated using the Fab preparation kit (Life Technologies, Canada). The anti-huFcγRIIIA 3G8 was from Biolegend, USA. Human serum albumin (HSA) was from Bayer, Canada. IVIg (Privigen) was from CSL Behring, Canada. Human serum IgG (huIgG, I4506) and bovine serum albumin (BSA) were from Sigma, Canada. The anti-CBC152 antibody was a kind gift from Dr. Uchikawa. The staining buffer used for flow cytometry was phosphate buffered saline (PBS) supplemented with 1% FBS, 1 mM EDTA adjusted to pH 7.4. The plasmids encoding the heavy and light chains of 6A6 IgG2a were gifts from Professor Falk Nimmerjahn at the University of Erlangen-Nuremberg, Germany.

Cloning and construction of fusion protein constructs. Total RNA was extracted from the 2.4G2 hybridoma using RNeasy™ kit (Qiagen, Hungary), and reverse transcription was initiated with oligo dT using RevertAid™ kit from Fermentas, Hungary. Combinations of forward and reverse primers (see Table below) were tested to identify best fitting sequences judged by the intensity and correct size of the polymerase chain reaction (PCR) product. VL sequence was amplified by the VκBackNco-VκFor4 pair, VH was obtained by VH5Cut-γCH3. PCR products were sequenced to confirm correct protein coding framework. Restriction endonuclease sites and linker sequence were introduced during a second PCR step, followed by overlapping extension PCR joining the VL and VH fragments. The final 2.4G2 scFv construct had the arrangement VL-(G₄S)₃-VH.

| | | SEQ |
|---|---|---|
| Internal | | ID |
| designation | Nucleic acid sequence | NO. |
| VκBackNco | TCC ATG GAC ATT GAG CTC ACC | 1 |
| | CAG TCT CC | |
| VκFor4 | TTT GAT TTC CAC CTT GGT CCC | 2 |
| VH5Cut | CAG GTA CAG CTA GTG GAG TCT GG | 3 |
| γCH3 | GGA TAG ACA GAT GGG GCT GTT G | 4 |

The 3G8 scFv sequence was kindly provided by Dr. Jörg Brünke (University of Erlangen-Nuremberg, Germany). The 3G8 scFv-MSA construct consists of the huFcγRIIIA -binding domain (3G8 scFv in the arrangement of VL-(G₄S)₄-VH) fused to human serum albumin (Uniprot P02768) via a hexa-glycine linker. The 2.4G2 scFv-MSA construct consists of the murine FcγRIII/IIB-binding domain (2.4G2 scFv in the arrangement of VL-(G4S)3-VH) fused to mouse serum albumin (MSA) (Uniprot P07724) via a hexa-glycine linker. Genes containing nucleotide sequences of the 3G8 scFv-HSA or the 2.4G2 scFv-MSA fusion construct were synthesized by GeneArt, USA. The constructs were then cloned into the mammalian expression vector pHLSec encoding a hexahistidine tag using AgeI and KpnI (New England Biolabs, Canada) as described (Yu et al. 2013). The soluble domain of huFcγRIIIA (of the high affinity valine 158 variant) was cloned into pHLSec as previously described (Yu et al. 2013). The nucleotide sequences of all constructs were verified by sequencing (ACGT Corp, Canada).

Recombinant protein expression and purification. The 3G8 scFv-HSA, 2.4G2 scFv-MSA, huFcγRIIIA and 6A6-IgG2a were all expressed by transient expression in HEK293T cells (a gift from Professor Jean-Philippe Julien, University of Toronto, Canada) in a similar fashion as previously described (Yu et al. 2013, Yu et al. 2015). Briefly, cells were grown to 90% confluence before transfection with polyethyleneimine and switched to serum free DMEM media (GE Healthcare, Canada) during recombinant protein expression. Cell culture supernatant was harvested 5 days after transfection and filtered (0.22 μm) before protein purification. Nickel sepharose and protein G agarose (both from GE Healthcare, Canada) were used to purify histidine-tagged recombinant proteins and 6A6-IgG2a respectively.

In vitro binding activity of 3G8 scFv-HSA. The binding activity of 3G8 scFv-HSA for huFcγRIIIA was assessed by enzyme-linked immunosorbent assay. The huFcγRIIIA was coated onto high-binding microtitre plates (Corning, 3590, Canada) at 5 μg/mL overnight at 4° C. High binding plates are designed to allow maximal adsorption of antigen onto the well surface and are recommended for general enzyme-linked immunosorbent assays. To examine direct binding of 3G8 scFv-HSA for huFcγRIIIA, the plate was blocked using 1% Casein (Life Technologies, Canada) for 1 hour, followed by incubation of serial dilutions of 3G8 scFv-HSA or HSA (highest concentration: 870 nM) for 1.5 hours at room temperature. Bound 3G8 scFv-HSA was detected by anti-human serum albumin-HRP (Abcam, Canada). To examine the ability of 3G8 scFv-HSA to inhibit huIgG binding to huFcγRIIIA, the plate was first blocked with 5% BSA, and then serial dilutions of 3G8 scFv-HSA, HSA (both highest concentration: 650 nM), or 3G8 (highest concentration: 67 nM) was added to wells containing 0.8 μg/mL huIgG. HuIgG was pre-mixed with these inhibitors before being adding to wells coated with huFcγRIIIA and allowed to bind for 1.5 hours at room temperature. Bound huIgG was detected by goat F(ab′)2 anti-human IgG (Fab′)2-HRP (Abcam, Canada). The 3,3′,5,5′-tetramethylbenzidine substrate (Life Technologies, Canada) was used for color development, and color development was stopped by adding 2 M H₂SO₄. Absorbance was measured at 450 nm on a Spectramax M5™ plate reader (Molecular Devices, Calif., USA).

In vitro binding activity of 2.4G2 scFv-MSA to RAW264.7 macrophages. RAW264.7 macrophage-like culture cells (ATCC, USA), known to express FcγRIIIA and FcγRIIB20, were used to examine the in vitro specificity of 2.4G2 scFv-MSA. To examine the direct binding of 2.4G2 scFv-MSA to RAW264.7 cells, 5×10⁵ cells were incubated with 0.11 μM 2.4G2 scFv-MSA (10 μg/ml) in the presence of the vehicle control (PBS) or an equimolar amount of 2.4G2 and HSA (as competitive inhibitors) for 1 hour on ice. The remaining bound 2.4G2 scFv-MSA was detected by anti-His-PE (Miltenyi Biotech, Canada). To examine the ability of 2.4G2 scFv-MSA to inhibit the binding activity of its parent antibody 2.4G2, 0.11 μM (10 μg/ml) 2.4G2 scFv-MSA, 2.4G2, or HSA was added to 5×10⁵ RAW264.7 cells in the presence of 0.013 μM (2 μg/ml) PE-labeled 2.4G2 (BD Biosciences, Canada) for 1 hour on ice; and residual bound 2.4G2-PE was quantified. MACS Quant flow cytometer (Miltenyi Biotech, Canada) was used for flow cytometry analysis and all data were processed by Flowjo V10 software (Flowjo, USA).

In vivo pharmacokinetics. To examine and compare the in vivo pharmacokinetics of 2.4G2 scFv-MSA and 2.4G2 Fab fragment, mice were injected intravenously with either 80 μg 2.4G2 scFv-MSA or approximately 200 μg 2.4G2 Fab. The molar ratio of 2.4G2 Fab to 2.4G2 scFv-MSA is approximately 4.5 to 1. These doses were selected to allow clear detection of residual 2.4G2 scFv-MSA and 2.4G2 Fab in serum 30 minutes after injection. Mice were bled 10 μl blood via the saphenous vein 0.5, 2, 4, 8, 24 and 48 hours after injection. The serum from each time point was prepared by centrifugation and stored at −80° C. before analysis. To examine the residual level of 2.4G2 scFv-MSA and 2.4G2 Fab after each time point, 2.5×10⁵ RAW264.7 cells were stained with 1/50 diluted serum for 1 hour. Bound 2.4G2 scFv-MSA was detected by anti-His-PE, and bound 2.4G2 Fab was detected by anti-rat IgG-κ chain-PE (Biolegend, USA). MACS Quant flow cytometer was used to analyze stained cell samples and all data were processed by Flowjo V10 software.

ITP induction and therapeutic treatment. All treatments were administered intravenously via the lateral tail vein unless otherwise stated. To examine the in vivo therapeutic effect of 2.4G2 scFv-MSA, mice were pre-treated with 10, 20, 40 or 80 μg of 2.4G2 scFv-MSA, 56 μg HSA (equimolar to 80 μg 2.4G2 scFv-MSA), or 25 mg IVIg (intraperitoneally) for 2 hours before induction of ITP by treatment of 2 μg MWReg30 or 3 μg 6A6-IgG2a. Mice were bled via the saphenous vein before treatment, then at 2, 24 and 48 hours after ITP induction, and the platelet number was enumerated by a Z2 particle counter (Beckman Coulter, Canada) as previously described (Yu et al. 2015).

Body temperature measurement. Body temperature was used to assess the occurrence of an anaphylactic response induced by different treatments (Khodoun et al. 2013, Iwamoto et al. 2015). Briefly, mice were injected intravenously with 0.43 nmol (65 μg) 2.4G2 or equimolar amount of 2.4G2 scFv-MSA or HSA. To crosslink 2.4G2 scFv-MSA before in vivo administration, half-molar amount of anti-His antibody was added to 0.43 nmol 2.4G2 scFv-MSA and incubated for 30 minutes at room temperature. Body (rectal) temperature was monitored 0.5, 1, 1.5 and 2 hours post-treatment using Thermocouple Thermometer, model TK-610B (Harvard Apparatus, USA).

Basophil quantification and CD200R3 detection. The level of CD200R3 expression on basophils from peripheral blood was examined using flow cytometry as described (Iwamoto et al. 2015, Nei et al. 2013). Briefly, mice were bled before treatment, 4 and 24 hours after treatment. RBCs were lysed by incubation with ammonium chloride buffer for 5 minutes at 37° C., and the peripheral blood mononuclear cells (PBMCs) were then stained with anti-CD49b-Pacific Blue (DX5), anti-FcεRIα-PerCP/Cy5.5 (MAR-1) (both from Biolegend, USA), and anti-CD200R3-FITC (BA103) (Hycult Biotech, Netherland). Basophils were gated as FcεRIα positive, CD49b dim cells, and confirmed with CD200R3 expression. The control blood basophil concentrations calculated in this experiment were compared against previous reports ensuring that the range is normal (Lantz et al. 2008, Hill et al. 2012).

Statistical analysis. The unpaired, two-tailed student t test was used to assess statistical significance between two data points throughout the study. GraphPad PRISM, Version 6.02 (GraphPad Software, Inc., La Jolla, Calif.) was used for data analysis.

HuFcγRIIIA-specific monovalent HSA fusion protein inhibits huIgG binding to huFcγRIIIA. To investigate whether a monovalent 3G8 fused to albumin would retain its specificity, we generated the 3G8 scFv-HSA fusion protein and demonstrated its target specificity towards huFcγRIIIA (FIG. 1A). Moreover, its ability to inhibit the interaction between huIgG and huFcγRIIIA was examined. As expected, 3G8 scFv-HSA was able to inhibit the binding of huIgG to huFcγRIIIA in a dose-dependent manner (FIG. 1B). The inhibitor constants for 3G3 and 3G8-scFv-MSA are approximately 1 nM and 40 nM respectively (FIG. 1B), demonstrating lowered binding efficiency of 3G8-scFv-MSA compared with its parent antibody 3G8 (FIG. 1B), likely as a result of reduced multivalency and protein domain rearrangement 24-26.

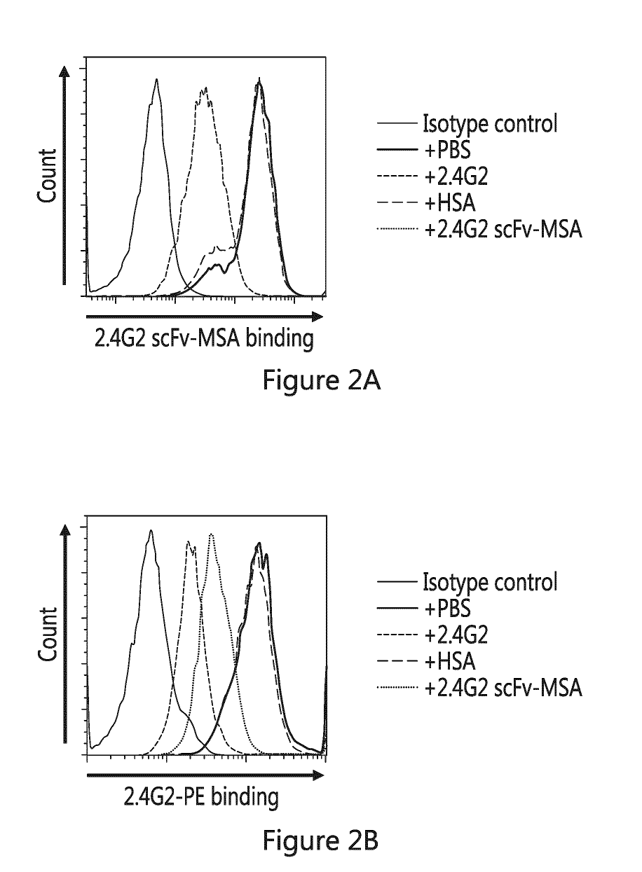

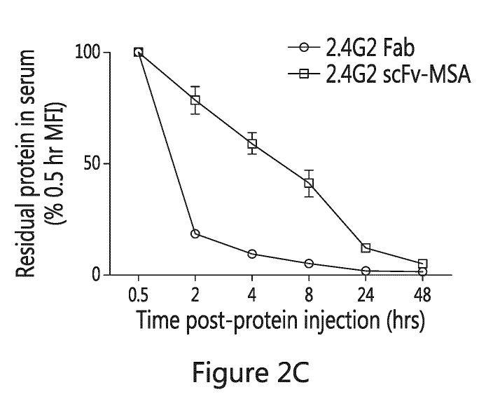

Monovalent 2.4G2 scFv-MSA fusion protein targets murine FcγRIII/IIB and exhibits favorable in vivo pharmacokinetics. To investigate the in vivo efficacy and adverse event profile of monovalent targeting, the 2.4G2 scFv-MSA fusion protein was generated, the murine counterpart of 3G8 scFv-HSA that targets murine FcγRIII/IIB. The RAW264.7 macrophage-like cell line is known to express murine FcγRIII/IIB20. The 2.4G2 scFv-MSA fusion protein was able to bind RAW264.7 cells (FIG. 2A), and its binding activity could be inhibited by the parent 2.4G2 antibody, but not by HSA (FIG. 2A). Conversely, the direct binding of 2.4G2 could be inhibited by 2.4G2 scFv-MSA and not by HSA (FIG. 2B). Consistent with the reduced affinity exhibited by the human 3G8 scFv-HSA (FIG. 1B), the parent 2.4G2 antibody displayed greater affinity than 2.4G2 scFv-MSA, evidenced by its superior ability to inhibit PE-labeled 2.4G2 binding to RAW264.7 cells (FIG. 2B). After establishing 2.4G2 scFv-MSA target specificity, in vivo pharmacokinetics was assessed in comparison with the 2.4G2 Fab, another monovalent molecule. As expected, the large size and lasting property of MSA enabled 2.4G2 scFv-MSA to exhibit superior pharmacokinetics in vivo compared with 2.4G2 Fab (FIG. 2C). Notably, approximately 80% of 2.4G2 Fab was cleared within 2 hours of administration, whereas 2.4G2 scFv-MSA stayed higher throughout all time points studied (FIG. 2C). Previous findings show that the half-life of albumin in humans is approximately 13-18 days, whereas that of mice is approximately 1 day. The findings in this in vivo pharmacokinetics study are therefore consistent with previous reports and support the establishment that the half-life of albumin in mice is shorter than humans.

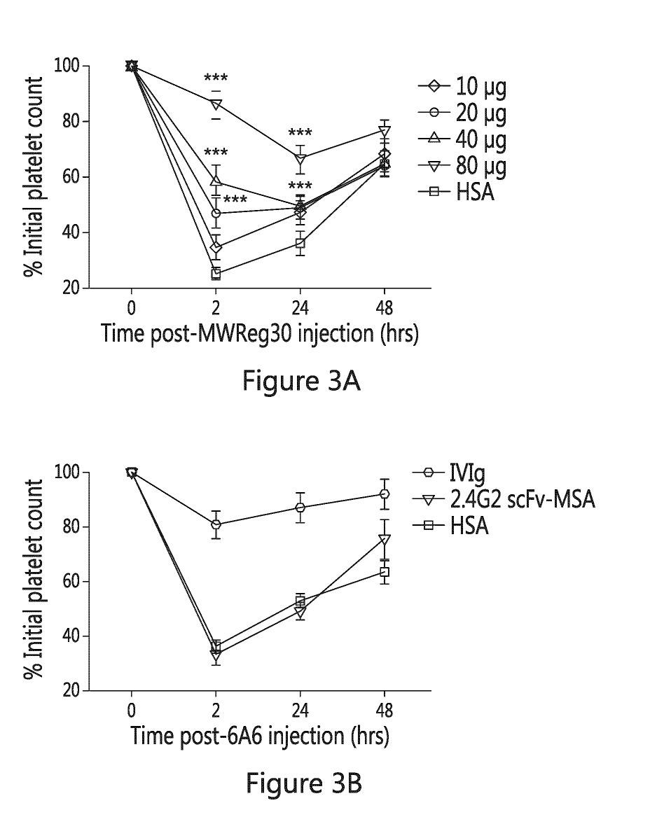

2.4G2 scFv-MSA inhibits FcγRIII, but not FcγRIV-mediated ITP. After establishing the target specificity and favorable pharmacokinetics, we next investigated the efficacy of 2.4G2 scFv-MSA in ITP amelioration. The anti-platelet antibody MWReg30 is known to mediate platelet clearance predominantly through FcγRIII30,31, a target of 2.4G2 scFv-MSA. Pretreatment with 2.4G2 scFv-MSA for 2 hours before ITP induction by MWReg30 resulted in significantly higher platelet counts compared with the control (FIG. 3A). Moreover, this ITP ameliorative effect was dose-dependent (FIG. 3A). Furthermore, the therapeutic effect of 2.4G2 scFv-MSA was maximal 2 hours post anti-platelet antibody injection (i.e. 4 hours after initial injection of 2.4G2 scFv-MSA), and declined 24 hours post injection (FIG. 3A). This diminutive trend over time correlates with the in vivo pharmacokinetics of 2.4G2 scFv-MSA (FIG. 2C), consistent with the fact that MSA has a much shorter half-life as compared to larger primates. To further confirm the in vivo specificity of 2.4G2 scFv-MSA, another anti-platelet antibody 6A6 (of the murine IgG2a isotype) was employed, it is known to mediate platelet depletion via FcγRIV32. It was found that 80 μg 2.4G2 scFv-MSA significantly ameliorated MWReg30-induced ITP (FIG. 3A), had no effect on 6A6-mediated platelet depletion (FIG. 3B) and demonstrated the expected in vivo specificity of 2.4G2 scFv-MSA.

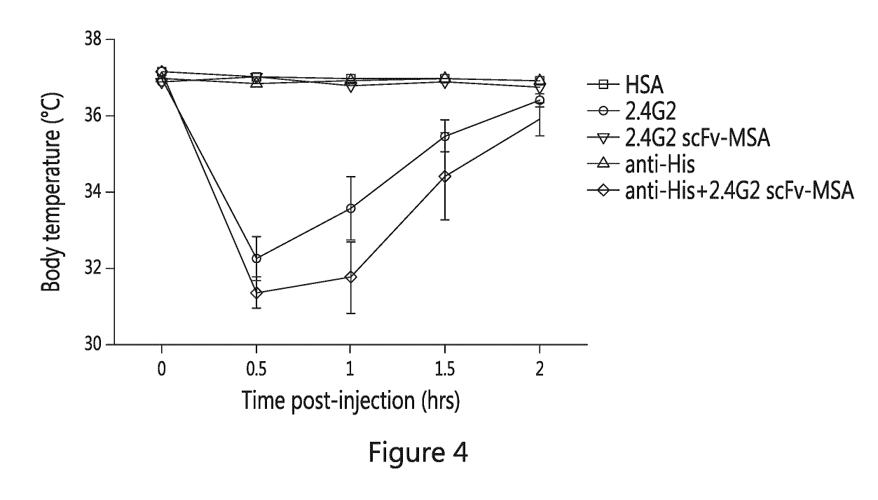

The parent antibody 2.4G2, not 2.4G2 scFv-MSA, triggers body temperature decrease. After establishing the in vivo efficacy of 2.4G2 scFv-MSA, it was then examined whether 2.4G2 scFv-MSA induces in vivo adverse events. Consistent with previous reports, administration of 0.43 nmol (65 μg) 2.4G2 triggered a rapid drop in the body temperature of mice, which was recovered by 2 hours (FIG. 4). A similar decrease in body temperature was absent when mice were treated with 2.4G2 scFv-MSA or HSA (FIG. 4). To investigate whether reversing the monovalency of 2.4G2 scFv-MSA would recapitulate the drop in body temperature, we used a monoclonal anti-His antibody to crosslink 2.4G2 scFv-MSA. Treatment with a crosslinked preparation of 2.4G2 scFv-MSA induced a similar drop in body temperature as compared to the parent 2.4G2 antibody (FIG. 4).

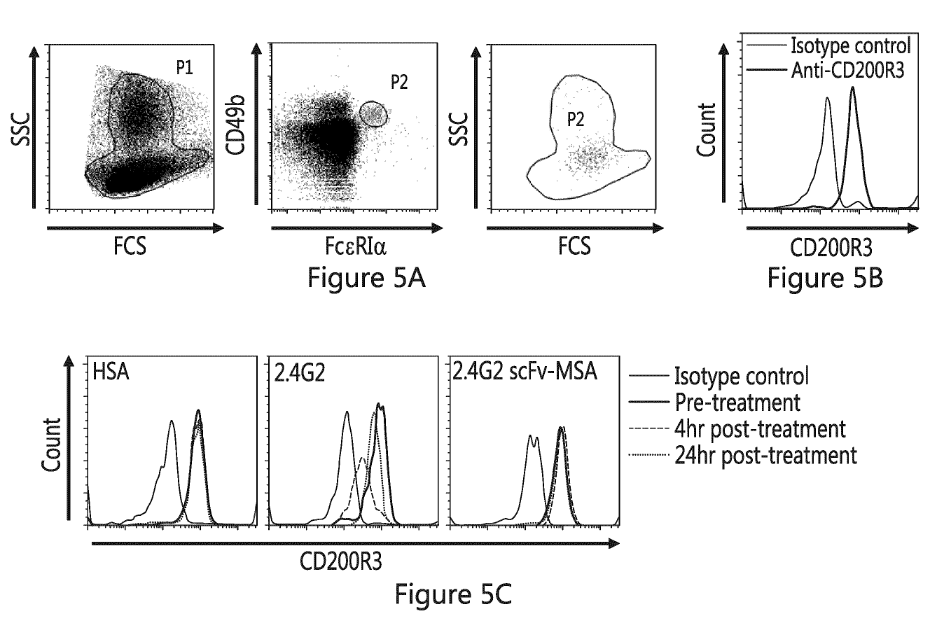

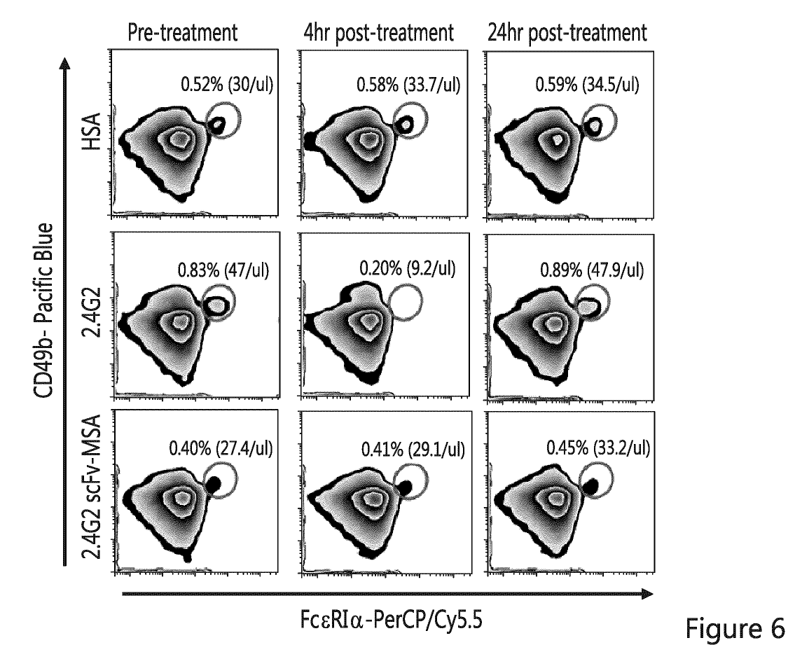

Antibody 2.4G2-induced basophil activation and depletion is absent in response to 2.4G2 scFv-MSA. In addition to changes in body temperature, the basophil activation-related marker CD200R3 was examined. A recent report demonstrated that 2.4G2-induced anaphylaxis significantly reduced basophil expression of CD200R3, an activating cell surface receptor. CD200R3 was expressed on basophils (FIGS. 5A-B). The administration of 0.43 nmol (65 μg) 2.4G2 rapidly reduced the ability to detect CD200R3 on basophils, which partially recovered after 24 hours (FIG. 5C). In contrast, neither 2.4G2 scFv-MSA nor HSA significantly modulated CD200R3 levels. In addition to CD200R3 expression, a transient basophil depletion in response to 2.4G2 administration was observed, which was also largely recovered after 24 hours (FIG. 6). In contrast, both 2.4G2 scFv-MSA and HSA had no significant effect on blood basophil levels (FIG. 6).

Fc receptor blockade has long been considered a viable strategy to treat antibody-mediated platelet destruction. Some existing ITP therapeutics, such as anti-D and IVIg, have been speculated to include a level of Fc receptor blockade in their modes of action. The huFcγRIIIA-specific mAb 3G8 was first described in 1982 and shown to improve ITP in refractory patients. The effective reversal of the low platelet count by the first anti-huFcγRIIIA antibody, 3G8, suggested the possibility of superseding current plasma-derived therapeutics with a monoclonal substitute. However, the clinical adverse events encountered during the pilot trials forestalled further development. While the exact cause of these adverse events remains unclear, a main potential mechanism involves the multivalent crosslinking of the activatory FcγRIIIA, mediated by the antigen-binding domain and Fc domain of the antibody. Based on this theory, a second generation anti-huFcγRIIIA antibody, GMA161, engineered to lack Fc-mediated FcγR engagement, had been developed. However, GMA161 failed to arrest the adverse events in refractory ITP patients, pointing out the genesis of these adverse events by some other attribute of the therapeutic antibody. In this example, this adverse event profile was at least partially attributed to the bivalent antigen-binding domain of anti-FcγR antibodies.

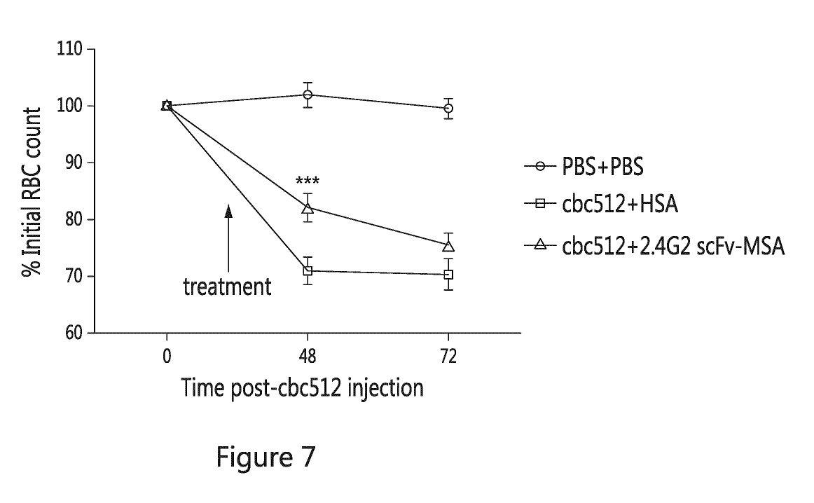

Monovalent 2.4G2 scFv-MSA fusion protein improves CDC512-mediated AHA. A mouse model of autoimmune hemolytic anemia (HOD mice injected with anti-CDC512 antibodies) was used to determine if the monovalent 2.4G2 scFv-MSA could limit the progression of the disease. The hemolytic anemia was first induced by injecting anti-CDC512 antibodies. Then, 24 hours later, the monovalent 2.4G2 scFv-MSA or HSA were administered. The red blood cell count was enumerated 48 and 72 hours after the induction of the anemia. As shown on FIG. 7, the administration of the monovalent 2.4G2 scFv-MSA prevented some of the hemolysis induced by the administration of anti-CDC512.

Activating FcγRs can normally be crosslinked by the IgG Fc, typically by the formation of immune complexes, to initiate an immune response. Such coordinated FcγR crosslinking is crucial for antibody-mediated immune function. However, uncontrolled crosslinking, as occurs upon the injection of anti-FcγR antibodies, could lead to undesired adverse events, demonstrated by the trials of 3G8 and GMA1618,11. Such anti-FcγR antibody-induced anaphylaxis is reminiscent of systemic inflammation triggered by certain pathological superantigens. To overcome the multivalency intrinsic to an anti-FcγR antibody a monovalent approach was developed in an attempt to circumvent the adverse events whilst retaining therapeutic efficacy. A fusion protein (3G8 scFv-HSA) composed of a single huFcγRIIIA-binding domain of 3G8 fused to HSA was generated and retained the ability to bind huFcγRIIIA and inhibit IgG-huFcγRIIIA interaction.

Next, to investigate the in vivo feasibility of such a monovalent approach, we generated a fusion protein (2.4G2 scFv-MSA) composed of a single FcγRIII-binding domain of 2.4G2 fused to MSA, and demonstrated its therapeutic efficacy in a passive murine ITP model. Moreover, 2.4G2 scFv-MSA was shown to successfully overcome the 2.4G2 antibody-induced body temperature decrease, a common measure of anaphylaxis. Importantly, it was also demonstrated that by crosslinking 2.4G2 scFv-MSA, the decrease in body temperature was recapitulated, further supporting a major role of multivalent crosslinking in causing adverse events. In addition to body temperature, 2.4G2 scFv-MSA lacked the ability to activate basophils demonstrated by its parent 2.4G2 antibody. Basophils are known to be pivotal for IgG-induced anaphylaxis, and the basophilic surface receptor CD200R3 has recently been demonstrated to be a marker for anti-FcγR antibody-mediated anaphylaxis. The finding that the 2.4G2-mediated anaphylactic response significantly lowered CD200R3 levels on basophils, an effect absent in response to 2.4G2 scFv-MSA treatment, was confirmed. In addition to the decreased CD200R3 level, a transient basophil depletion in response to 2.4G2 treatment, but not 2.4G2 scFv-MSA, was observed. Murine basophils are known to express significant levels of FcγRIII39, and thus would be a target for 2.4G2-mediated depletion. This transient depletion is consistent with the anti-huFcγRIIIA GMA161-induced granulocyte depletion in the humanized mouse model.

The 2.4G2 scFv-MSA exhibited superior pharmacokinetics compared with the monovalent Fab fragment, likely as a result of its larger size and the extended half-life of albumin. Indeed, in recent years, significant progress has been made to prolong the half-life of protein-based therapeutics, culminating in the approval of several clinical products Some notable strategies include increasing the size of the protein or conferring binding affinity to the FcRn, a receptor conferring extended half-life of IgG and albumin. Albumin-coupled therapeutics have recently entered the list of approved medicines, further supporting the feasibility of this albumin fusion protein. Although 2.4G2 scFv-MSA exhibited significantly improved pharmacokinetics compared to its Fab counterpart, approximately 90% was cleared within the first 24 hours, raising the issue of short-lasting in vivo efficacy. Previous studies have conclusively established that the in vivo longevity of albumin is directly proportional to the size of the animal, with mice having the shortest half-life. This shorter half-life of albumin in mice prevented us from establishing a therapeutic ITP mouse model, as such a model requires the treatment to typically stay in circulation for two days to enable detection of the therapeutic effects. Thus, independent models involving larger animals could help investigate its efficacy in an active ITP or other FcγR-implicated diseases.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Hill D A, Siracusa M C, Abt M C, et al. Commensal bacteria-derived signals regulate basophil hematopoiesis and allergic inflammation. Nat Med. 2012; 18 (4):538-546.

Hiroshi Iwamoto* T M, Yuki Nakazato, Kazuyoshi Namba and Yasuhiro Takeda. Decreased expression of CD200R3 on mouse basophils as a novel marker for IgG1-mediated anaphylaxis. Immunity, Inflammation and Disease. 2015.

Khodoun M V, Kucuk Z Y, Strait R T, et al. Rapid desensitization of mice with anti-FcgammaRIIb/FcgammaRIII mAb safely prevents IgG-mediated anaphylaxis. J Allergy Clin Immunol. 2013; 132 (6):1375-1387.

Lantz C S, Min B, Tsai M, Chatterjea D, Dranoff G, Galli S J. IL-3 is required for increases in blood basophils in nematode infection in mice and can enhance IgE-dependent IL-4 production by basophils in vitro. Lab Invest. 2008; 88 (11):1134-1142.

Nei Y, Obata-Ninomiya K, Tsutsui H, et al. GATA-1 regulates the generation and function of basophils. Proc Natl Acad Sci U S A. 2013; 110 (46):18620-18625.

Yu X, Baruah K, Harvey D J, et al. Engineering hydrophobic protein-carbohydrate interactions to fine-tune monoclonal antibodies. J Am Chem Soc. 2013.

Yu X, Menard M, Seabright G, Crispin M, Lazarus A H. A monoclonal antibody with anti-D-like activity in murine immune thrombocytopenia requires Fc domain function for immune thrombocytopenia ameliorative effects. Transfusion. 2015; 5 5(6 Pt 2):1501-1511.

1. A monovalent antibody moiety for limiting or avoiding the activation of an immune cell caused by the presence and the binding of a ligand of an activating Fc receptor to the activating Fc receptor, the monovalent antibody moiety:
lacking a Fc region; and
being capable of specifically binding to a component of an activating Fc receptor.

2. The monovalent antibody moiety of claim 1 being a competitive inhibitor of the activating Fc receptor.

3. The monovalent antibody moiety of claim 1 being a single chain variable fragment (scFv).

4. The monovalent antibody moiety of claim 1 being a fragment antigen-binding (Fab).

5. The monovalent antibody moiety of claim 1 being derived from a 3G8 antibody.

6. The monovalent antibody moiety of claim 1 being derived from a 2.4G2 antibody.

7. The monovalent antibody moiety of claim 1, wherein the activating Fc receptor is a FcγR receptor.

8. The monovalent antibody moiety of claim 1, wherein the activating Fc receptor is a FcγRIII polypeptide.

9. A chimeric protein comprising the monovalent antibody moiety of claim 1 and a carrier, wherein the carrier is physiologically acceptable, lacks the ability to induce a pro-inflammatory immune response and has a molecular weight equal to or greater than 40 kDa.

10. The chimeric protein of claim 9, wherein the monovalent antibody moiety is covalently associated to the carrier.

11. The chimeric protein of claim 9, further comprising a linker between the monovalent antibody moiety and the carrier.

12. The chimeric protein of claim 11, wherein the linker is an amino acid linker.

13. The chimeric protein of claim 9, wherein the carrier is a polypeptide.

14. The chimeric protein of claim 13, wherein the polypeptide is albumin.

15. The chimeric protein of claim 9, wherein the carboxyl terminus of the monovalent antibody moiety is associated to the carrier.

16. The chimeric protein of claim 15, wherein the carrier is a polypeptide and the amino terminus of the carrier is associated to the carboxyl terminus of the monovalent antibody moiety.

17.-20. (canceled)

21. A method for preventing, treating or alleviating the symptoms of an auto-immune inflammatory condition or disorder caused or maintained by the engagement of an auto-antibody having a Fc region capable of engaging with an activating Fc receptor in a subject in need thereof, said method comprising administering a therapeutically effective amount of a monovalent antibody moiety as defined in claim 1 so as to prevent, treat or alleviate the symptoms of the auto-immune inflammatory condition or disorder in the subject.

22. The method of claim 21, wherein the auto-immune inflammatory condition or disorder is immune thrombocytopenia.

23. The method of claim 22, wherein the immune thrombocytopenia is idiopathic.


SEQUENCE LISTING (4 sequences)

SEQ ID NO: 1 - DNA, 29 bp, Artificial Sequence
  Oligonucleotide
tccatggacattgagctcacccagtctcc

SEQ ID NO: 2 - DNA, 21 bp, Artificial Sequence
  Oligonucleotide
tttgatttccaccttggtccc

SEQ ID NO: 3 - DNA, 23 bp, Artificial Sequence
  Oligonucleotide
caggtacagctagtggagtctgg

SEQ ID NO: 4 - DNA, 22 bp, Artificial Sequence
  Oligonucleotide
ggatagacagatggggctgttg